(19) United States
(12) Patent Application Publication
(10) Pub. No.: US 2023/0001326 A1
(43) Pub. Date: Jan. 5, 2023

(54) PROCESS FOR EXTRACTING BIOACTIVE COMPOUNDS FROM PLANT MATERIALS

(71) Applicant: Winters Capital IP LLC, Alpharetta, GA (US)

(72) Inventors: Clint Winters, Alpharetta, GA (US); Travis Shoaf, Alpharetta, GA (US)

(21) Appl. No.: 17/847,195

(22) Filed: Jun. 23, 2022

Related U.S. Application Data
(60) Provisional application No. 63/217,959, filed on Jul. 2, 2021.

(51) Int. Cl. B01D 11/02; A61K 36/185

(52) U.S. Cl. CPC B01D 11/0288; A61K 36/185; A61K 2236/15; A61K 2236/35; A61K 2236/53

(57) ABSTRACT

A process of extracting bioactive compounds from one or more plant material is provided. The process includes agitation of one or more plant material in the presence of piperine and palmitoylethanolamide to produce a powder blend, supplying of a polar solvent to the powder blend and then adding a nonpolar solvent after at least 24 hours of infusion of the powder blend in the polar solvents to form a slurry. The slurry is further filtered to recover bioactive compounds in a liquid extract form.

### CROSS-REFERENCE TO RELATED APPLICATIONS

The present application is related to and claims benefit under 35 U.S.C. § 119(e) from U.S. Provisional Patent Application Ser. No. 63/217,959 filed on Jul. 2, 2021.

### BACKGROUND OF THE DISCLOSURE

The popularity and widespread use of plant extracts for various health benefits has risen in recent years. The plant extracts are extracted from plant materials such as flowers, leaves, bark, stems, roots, peels, trichomes using various extraction processes to obtain bioactive compounds for its medicinal, wellness and nutritional benefits. The bioactive compounds are also called phytochemicals or plant derived compounds and can be extracted using a liquid extraction process. The presently well known liquid extraction processes involve either polar or nonpolar solvent extraction to isolate specific bioactive compounds from the plant extract. Due to the larger and uneven surface area of powdered particles of the plant material, the high amount of the solvent may be used in these solvent extraction methods which further requires expensive operations to recover bioactive compounds. Combining the different plant extracts also requires separate extraction processes to achieve combine extract to avoid losing bioactive compounds during the process.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 is a schematic view of a process of an extraction in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION OF THE DISCLOSURE

An aspect of the specification provides a process for extracting bioactive compounds from a plant material, where the process includes isolating a plant material from a plant by grinding or cutting to a predetermined size , agitating the plant material in presence of piperine and palmitoylethanolamide to produce a powder blend, adding a polar solvent selected from the group consisting of food grade 200 proof ethyl alcohol, water, dimethylsulfoxide, acetone, acetonitrile, formaldehyde, ether, methanol, glacial acetic acid, dimethylformamide, hydrochloric acid, ethylene glycol, tetrahydrofuran, dichloromethane and combination thereof to the powder blend, thereby dissolving piperine and palmitoylethanolamide.

Process may furthermore include, adding a nonpolar solvent selected from the group consisting of d-limonene, hexane, toluene, xylene, heptane, pentane, ethyl acetate, chloroform, isopropyl alcohol, polyethylene glycol 400, and combination thereof, at least 24 hours after infusing the powder blend with the polar solvent, wherein a quantity ratio of the polar solvent to nonpolar solvent is 1:9. The process may in addition include agitating the powder blend, the polar solvent and the nonpolar solvent for at least 48 hours to produce a slurry and filtering the slurry to produce a liquid extract having the bioactive compounds with pH of about 7.0 to about 7.4.

The plant is selected from the group consist of Cannabis sativa, Cannabis indica or Cannabis ruderalis, Tabernaemontana divaricata, Withania somnifera, Euphausia pacifica, Euphausia superba, Haematococcus pluvialis, Pandalus borealis, Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof.

An another aspect of the specification provides a process for extracting bioactive compound, where the process includes isolating a first plant material from a cannabis plant and isolating a second plant material from a second plant from the group consisting of Tabernaemontana divaricata, Withania somnifera, Euphausia pacifica, Euphausia superba, Haematococcus pluvialis, Pandalus borealis , Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof. The first plant is selected from the group consisting of Cannabis sativa, Cannabis indica or Cannabis ruderalis and combination thereof.

The process also includes agitating the plant material and the second plant material in presence of piperine and palmitoylethanolamide to produce a powder blend, and adding a polar solvent selected from the group consisting of food grade 200 proof ethyl alcohol to the powder blend, thereby dissolving piperine and palmitoylethanolamide. The process may furthermore include adding a nonpolar solvent selected from the group consisting of d-limonene at least 24 hours after infusing the powder blend with the polar solvent, wherein a quantity ratio of the polar solvent to nonpolar solvent is 1:9. The process further includes agitating the powder blend, the polar solvent and the nonpolar solvent for at least 48 hours to produce a slurry and filtering the slurry to produce a liquid extract having the bioactive compounds with pH of about 7.0 to about 7.4.

Another aspect of the specification provides a liquid extract of bioactive compounds anti-inflammatory and analgesic properties with increased bioavailability. Another objective of the present specification is to provide non-cannabis cannabinoid compounds synergistically with other compounds to modulate the overall analgesic effects of said compound, thereby creating a more potent analgesic property.

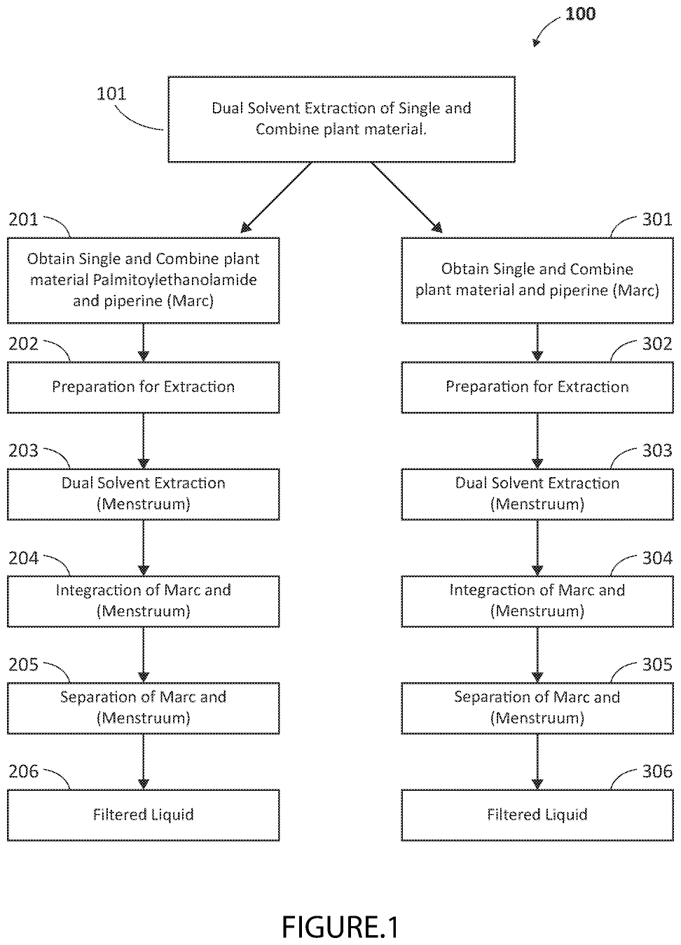

FIG. 1 illustrates a schematic view of a process 100 of an extraction in accordance with some embodiments. As illustrated, at operation 101 a plant material is isolated by grinding or cutting to a predetermined size, where the plant material can be selected from the plants including Cannabis sativa, Cannabis indica and Cannabis ruderalis, Tabernaemontana divaricata, Withania somnifera, Euphausia pacifica, Euphausia superba, Haematococcus pluvialis, Pandalus borealis , Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof.

According to another aspect, at operation 101, a first plant material can be selected from a cannabis plant and isolating a second plant material from a second plant from the group consisting of Tabernaemontana divaricata, Withania somnifera, Euphausia pacifica, Euphausia superba, Haematococcus pluvialis, Pandalus borealis , Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof. 291-1-USUTIL

The plant materials can be selected from various parts of the plant including the group consisting of roots, flowers, seeds, leaves, stem, trichomes, bark and roots of the cannabis plant. Once harvested, the plant material may be dried and powdered having an average particle size of about 50 μm to about 1 mm. The drying process reduces moisture content of the plant, typically around 75% moisture when harvested, to a resultant moisture content of 10-15%. Some of the bioactive compounds from cannabis plants can easily decompose when subjected to heat and ultraviolet (UV) radiation, hence natural drying processes may be used. The plant material can be powered by a grinding process. The operation 101 also includes verification of powdered material for presence of hazardous microbes and presence of dangerous levels of toxic metals. These tests are designed to be performed in order to assure that the material is accurate for identity and foreign particles and microorganisms will not affect the extraction process.

At operation 102, the powdered plant material is agitated in presence of piperine and palmitoylethanolamide powder(s) in order to create a more viable surface area of particles for the extraction process. The agitation with piperine and palmitoylethanolamide can be carried out in a ribbon blender for at least 20 minutes to produce a powder blend. Piperine is typically extracted from Piper nigrum, and can also be found in Piper longum and Piper officinarum and obtained in a powder form. Upon consumption piperine activates the heat- and acidity-sensing transient receptor potential channel ion channels, transient receptor potential cation channel subfamily V member 1 and A member 1 (TRPV1 and TRPA1), on nociceptors, the pain-sensing nerve cells. Palmitoylethanolamide is well known for its anti-inflammatory and analgesic properties and can be combined to achieve synergistic effects. The powder blend is then removed from the ribbon blender and can be placed into a sanitized, approved container for transport to a tincture batching room(s) in a way so as to avoid adulteration or cross contamination per good manufacturing practices standards.

At operation 103, the polar solvent can be added to the powder blend. The operation can be carried in a brew kettle placed on a hydraulic lift, wherein the powder blend is fed through a conveyor into a brew kettle placed and sprayed with the polar solvent. The brew kettle is subject to rigorous sanitization process prior to beginning the assembly of components in the following order: The disinfecting wipes can be used to wipe the interior and exterior of the brew kettle and lid in order to eliminate any errant microbials that could be present and to remove more visible potential adulterants.

The polar solvent can be selected form the group consisting of food grade 200 proof ethyl alcohol, water, dimethylsulfoxide, acetone, acetonitrile, formaldehyde, ether, methanol, glacial acetic acid, dimethylformamide, hydrochloric acid, ethylene glycol, tetrahydrofuran, dichloromethane and combination thereof to the powder blend and allow to infuse for at least 24 hours thereby dissolving piperine and palmitoylethanolamide.

At operation 104, the nonpolar solvent can be added to the powder blend. The nonpolar solvent can be selected from the group consisting of d-limonene, hexane, toluene, xylene, heptane, pentane, ethyl acetate, chloroform, isopropyl alcohol, polyethylene glycol 400, and combination thereof, wherein a quantity ratio of the polar solvent to nonpolar solvent is 1:9. The higher concentration of ethyl alcohol may destroy bioactive compounds hence a strict quantity ratio of 1:9 is to provide enough to slightly accelerate the extraction process and maintain the plant material's chemical integrity. The ethyl alcohol is also used in the pharmaceutical formulation to assist with absorption as it promotes quicker access through the mucous membrane when consumed and into the blood brain barrier, thereby allowing the active compounds to work more quickly. In addition, the piperine and palmitoylethanolamide are soluble in ethyl alcohol, hence the two components can be extracted prior to adding nonpolar solvents.

The non-polar solvents are less likely to affect the chemical structure of the plant material. Additionally, the nonpolar solvents like d-limonene are a derivative of citrus fruits, this solvent provides a pleasant flavor to the extracted liquid.

At operation 105, the powder blend, polar solvent and nonpolar solvent are agitated for at least 48 hours to produce a slurry. The agitation can be carried out to achieve a consistent and effective environment for extraction by allowing the surface area of the powder particles to be covered with the solvents. This can be achieved by agitating the solution until a homogeneous slurry is formed. Once the slurry is achieved a lid can be replaced atop the brew kettle in order to create a sealed environment for the extraction to take place. The slurry is left undisturbed for at least 24 hours before filtration. The lid is placed in a manner so as to avoid adulteration and contamination and left undisturbed for at least 24 total hours, and not more than 120 hours. This combined time frame of at least 48 hours can be utilized for the broad-spectrum dual solvent liquid extraction of bioactive compounds from the powder blend in order to achieve the proper efficacy and concentrations.

At operation 106, the slurry can be filtered to produce a liquid extract with a pH of about 7.0 to about 7.4. The filtration can be carried out using a siphoning of

the liquid extract from the brew kettle into a polyethylene terephthalate (PET) plastic container. The Siphoning can include but not limited to a 13.25″ sifting pan with mesh size of 1/100th″ or (0.01)″ affixed atop the plastic container. This filtration process allows the liquid extract to be separated from the undissolved powder blend.

This extraction process can also be referred to as a broad-spectrum dual solvent extraction process to extract possible bioactive compounds from the plant material. The bioactive compounds can include cannabidiol, delta-9-tetrahydrocannabinola identified in cannabis plants. There are more than 550 chemical compounds in cannabis, with more than 100 phytocannabinoids being identified, including Δ9-tetrahydrocannabinol (THC) and cannabidiol (CBD).

The bioactive compounds can include conolidine and indole alkaloids, showing analgesic effects can be isolated from bark of the Tabernaemontana divaricata. The bioactive compounds also include 66 alkaloids such as but not limited to conolidine, catharanthine, coronaridine, dregamine, ibogamine, tabersonine, voacamine and voacristine and combination thereof.

“Plant material” refers to any part of a plant, and includes bark, wood, leaves, stems, roots, flowers, fruits, seeds, berries as well as exudates, which optionally has been dried, cut into pieces, milled, or powdered. “Cannabis” refers to plants of the genus Cannabis. The genus includes Cannabis sativa, (sometimes divided into the two subspecies Cannabis indica and Cannabis ruderalis).

The extracts obtained as described above provide a basis for pharmaceutical compositions that comprise the extract alone or combined with other plant material and at least one pharmaceutically acceptable carrier, where the other plant material can be obtained from the from the group consisting of Cannabis sativa, Cannabis indica or Cannabis ruderalis, Tabernaemontana divaricata, Withania somnifera, Euphausia pacifica, Euphausia superba, Haematococcus pluvialis, Pandalus borealis, Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof.

The term “pharmaceutical composition” as used here includes cosmetic compositions, medicinal and nutraceutical compositions. The pharmaceutical compositions can be administered to the skin in the form of an ointment, a poultice, a plaster, a compress, a balm, an unguent, a salve, an emollient, and other forms suitable for administering substances to the skin for pain management and its analgesic effect. The pharmaceutical compositions can also be formulated for routes of administration including oral and sublingual.

The pharmaceutical compositions described herein can be administered to a human patient per se, or as part of other pharmaceutical compositions where they are mixed with other active ingredients, as in combination therapy, or suitable carriers or excipient(s).

The pharmaceutical compositions described herein can be used as hallucinogenic, hypnotic, sedative, analgesic, anti-inflammatory agents and combination thereof.

### EXAMPLES

### Example 1

Dried leaves and stems of Cannabis sativa (100 g) were milled to a fine powder, weighed into a glass container and mixed with dried leaves powder of Tabernaemontana divaricata plant material (100 g). The mixture then agitated in a ribbon blender for 20 minutes in presence of piperine (100 g) and palmitoylethanolamide (100 g) to produce a powder blend. The powder blend transferred through a conveyor into a sanitized brew kettle placed on a hydraulic lift and sprayed with food grade 200 proof ethyl alcohol (100 g) to dissolve piperine and palmitoylethanolamide. After 24 hours the infused powder blend was further sprayed with d-limonene (100 g). The mixture was further agitated for 48 hours to produce a slurry. The slurry was filtered to produce a liquid extract with a pH of about 7.4. The extraction process was carried out at the temperature of between 16° Celsius and 48° Celsius. The specific temperature was determined to avoid exceeding the flash point of the solvents. The assay was performed to identify bioactive compounds retired in the liquid extract. The liquid extract contained various phytochemicals including more than 66 alkaloids such as but not limited to conolidine, catharanthine, coronaridine, dregamine, ibogamine, tabersonine, voacamine and voacristine and combination thereof. The liquid extract was further allowed to cool and stored in a sealed glass container at ambient temperature.

### Example 2

Dried leaves and stems of Tabernaemontana divaricata (100 g) were milled to a fine powder, weighed into a glass container. The mixture then agitated in a ribbon blender for 20 minutes in presence of piperine (100 g) and palmitoylethanolamide (100 g) to produce a powder blend. The powder blend transferred through a conveyor into a sanitized brew kettle placed on a hydraulic lift and sprayed with food grade 200 proof ethyl alcohol (100 g) to dissolve piperine and palmitoylethanolamide. After 24 hours the infused powder blend was further sprayed with d-limonene (100 g). The mixture was further agitated for 48 hours to produce a slurry. The slurry was filtered to produce a liquid extract with a pH of about 7.4. The extraction process was carried out at the temperature of between 16° Celsius and 48° Celsius. The specific temperature was determined to avoid exceeding the flash point of the solvents. The assay was performed to identify bioactive compounds retired in the liquid extract. The liquid extract contained various phytochemicals including more than 66 alkaloids such as but not limited to conolidine, catharanthine, coronaridine, dregamine, ibogamine, tabersonine, voacamine and voacristine and combination thereof. The liquid extract was further allowed to cool and stored in a sealed glass container at ambient temperature.

In the foregoing specification, specific embodiments have been described. However, one of ordinary skill in the art appreciates that various modifications and changes can be made without departing from the scope of the specification as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The specification is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A process for extracting bioactive compounds comprising:
a) isolating a plant material from a plant by grinding or cutting to a predetermined size;
b) agitating the plant material in presence of piperine and palmitoylethanolamide to produce a powder blend;
c) adding a polar solvent selected from the group consisting of food grade 200 proof ethyl alcohol, water, dimethylsulfoxide, acetone, acetonitrile, formaldehyde, ether, methanol, glacial acetic acid, dimethylformamide, hydrochloric acid, ethylene glycol, tetrahydrofuran, dichloromethane and combination thereof to the powder blend, thereby dissolving piperine and palmitoylethanolamide;
d) adding a nonpolar solvent selected from the group consisting of d-limonene, hexane, toluene, xylene, heptane, pentane, ethyl acetate, chloroform, isopropyl alcohol, polyethylene glycol 400, and combination thereof, at least 24 hours after infusing the powder blend with the polar solvent, wherein a quantity ratio of the polar solvent to nonpolar solvent is 1:9;
e) agitating the powder blend, the polar solvent and the nonpolar solvent for at least 48 hours to produce a slurry; and
f) filtering the slurry to produce a liquid extract having the bioactive compounds with pH of about 7.0 to about 7.4.

2. The process for extracting bioactive compounds of claim 1, wherein the plant is selected from the group consist of Cannabis sativa, Cannabis indica or Cannabis ruderalis, Tabernaemontana divaricata, Withania somnifera, Euphausia pacifica, Euphausia superba, Haematococcus pluvialis, Pandalus borealis , Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof.

3. The process for extracting bioactive compounds of claim 1, wherein the plant is Cannabis sativa.

4. The process for extracting bioactive compounds of claim 1, wherein the plant is Tabernaemontana divaricata.

5. The process for extracting bioactive compounds of claim 1, wherein the plant material selected from the group consist of roots, flowers, seeds, leaves, stem, trichomes, bark and roots of the plant.

6. The process for extracting bioactive compounds of claim 1, wherein the plant material is dried and powdered having an average particle size of about 50 μm to about 1 mm.

7. The process for extracting bioactive compounds of claim 1, wherein the plant material is agitated with piperine and palmitoylethanolamide in a ribbon blender for at least 20 minutes to produce the powder blend.

8. The process for extracting bioactive compounds of claim 1, wherein the powder blend is fed through a conveyor into a brew kettle placed on a hydraulic lift, and is sprayed with the polar solvent and the nonpolar solvent, wherein the powder blend, the polar solvent and the nonpolar solvent is agitated for at least 48 hours to produce the slurry.

9. The process for extracting bioactive compounds of claim 1, wherein the process is a broad-spectrum dual solvent extraction, wherein the polar solvent is food grade 200 proof ethyl alcohol and the nonpolar solvent is d-limonene.

10. The process for extracting bioactive compounds of claim 1, wherein the process is carried out at a temperature between 16° Celsius and 48° Celsius.

11. The process for extracting bioactive compounds of claim 1, wherein the liquid extract is provided with the bioactive compounds with pH of about 7.4.

12. The process for extracting bioactive compounds of claim 1, wherein the slurry is left undisturbed for at least 24 hours before filtration.

13. A process for extracting bioactive compounds comprising:
a) isolating a first plant material from a cannabis plant;
b) isolating a second plant material from a second plant from the group consisting of Tabernaemontana divaricata, Withania somnifera, Euphausia pacifica, Euphausia superba, Haematococcus pluvialis, Pandalus borealis, Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma longifolia, Valeriana officinalis and Cornu Cervi Pantotrichum and combination thereof;
c) agitating the first plant material and the second plant material in presence of piperine and palmitoylethanolamide to produce a powder blend;
d) adding a polar solvent selected from the group consisting of food grade 200 proof ethyl alcohol, water, dimethylsulfoxide, acetone, acetonitrile, formaldehyde, ether, methanol, glacial acetic acid, dimethylformamide, hydrochloric acid, ethylene glycol, tetrahydrofuran, dichloromethane and combination thereof to the powder blend, thereby dissolving piperine and palmitoylethanolamide;
e) adding a nonpolar solvent selected from the group consisting of d-limonene, hexane, toluene, xylene, heptane, pentane, ethyl acetate, chloroform, isopropyl alcohol, polyethylene glycol 400, and combination thereof, at least 24 hours after infusing the powder blend with the polar solvent, wherein a quantity ratio of the polar solvent to nonpolar solvent is 1:9;
f) agitating the powder blend, the polar solvent and the nonpolar solvent for at least 48 hours to produce a slurry;and
g) filtering the slurry to produce a liquid extract having the bioactive compounds with pH of about 7.0 to about 7.4.

14. The process for extracting bioactive compounds of claim 13, wherein the cannabis plant is selected from the group consist of Cannabis sativa, Cannabis indica or Cannabis ruderalis.

15. The process for extracting bioactive compounds of claim 13, wherein the first plant material and the second plant material selected from the group consist of roots, flowers, seeds, leaves, stem, trichomes, bark and roots of the cannabis plant and the second plant from the group consisting of Tabernaemontana divaricata, Withania somnifera, Euphausia pacifica, Euphausia superba, Haematococcus pluvialis, Pandalus borealis , Astragalus propinuus, Bacopa monnieri, Beta vulgaris, Berberis vulgaris, Brown algae, Curcuma longa, Camellia sinensis, Centella asiatica, Sambucus nigra, Crataegus monogyna, Reynoutria japonica, Panax ginseng, Melissa officinalis, Hericium erinaceus, Pinus tabuliformis, Punica granatum, Serenoa repens, Eurycoma Valeriana officinalis and Cornu Cervi Pantotrichum.

16. The process for extracting bioactive compounds of claim 13, wherein the first plant material and the second plant material is dried and powdered having an average particle size of about 50 μm to about 1 mm.

17. The process for extracting bioactive compounds of claim 13, wherein the first plant material and the second plant material is agitated with piperine and palmitoylethanolamide in a ribbon blender for at least 10 minutes to produce the powder blend.

18. The process for extracting bioactive compounds of claim 13, wherein the powder blend is fed through a conveyor into a brew kettle placed on a hydraulic lift, and is sprayed with the polar solvent and the nonpolar solvent, wherein the powder blend, the polar solvent and the nonpolar solvent is agitated for at least 48 hours to produce the slurry.

19. The process for extracting bioactive compounds of claim 13, wherein the process is a broad-spectrum dual solvent extraction, wherein the polar solvent is food grade 200 proof ethyl alcohol and the nonpolar solvent is d-limonene.

20. The process for extracting bioactive compounds of claim 13, wherein the process is carried out at a temperature between 16° Celsius and 48° Celsius.

21. The process for extracting bioactive compounds of claim 13, wherein the liquid extract is provided with the bioactive compounds with pH of about 7.4.

22. The process for extracting bioactive compounds of claim 13, wherein the slurry is left undisturbed for at least 24 hours before filtration.
